(19) United States
(12) Patent Application Publication
(10) Pub. No.: US 2010/0323436 A1
(43) Pub. Date: Dec. 23, 2010

(54) PHOTOBIOREACTOR FOR LARGE-SCALE CULTURE OF MICROALGAL

(76) Inventors: Choul-Gyun Lee, Seoul (KR); Z-Hun Kim, Incheon (KR)

Correspondence Address: MAIER & MAIER, PLLC, 1000 DUKE STREET, ALEXANDRIA, VA, 22314, US

(21) Appl. No.: 12/516,112

(22) PCT Filed: Nov. 28, 2008
(86) PCT No.: PCT/KR08/07046

§ 371 Date: May 22, 2009

(30) Foreign Application Priority Data
Nov. 28, 2007 (KR) 10-2007-0121948
Nov. 28, 2007 (KR) 10-2007-0121949

(51) Int. Cl. C12M 1/00

(52) U.S. Cl. 435/292.1

(57) ABSTRACT

A photobioreactor for a large-scale microalgal culture. The photobioreactor includes a plurality of surface-light-source assemblies using a light emitting diode (LED) element, an organic light emitting diode (OLED) element, or a flexible LED sheet as a light source. The surface-light-source assemblies have the shape of a flat plate or a cylinder, are installed at predetermined intervals in the interior space of a reaction tank having the shape of a cube or a cylinder, and emit light for culturing microalgae in an internally illuminated fashion. Here, the surface-light-source assemblies are installed in such a manner that they are alternately in contact with first and second walls of the reaction tank disposed in parallel to each other in a symmetrical shape, and serve as partitions that portion the interior space of the reaction tank and increase a flow distance of gas.

### CROSS-REFERENCES TO RELATED APPLICATION

This patent application claims the benefit of priority under 35 U.S.C. §119 from Korean Patent Application No. 10-2007-0121948 and No. 10-2007-0121949 filed on Nov. 28, 2007, the contents of which are incorporated herein by reference.

### BACKGROUND OF THE INVENTION

1. Field of the Invention

The present invention relates, in general, to a photobioreactor for a large-scale microalgal culture, and more particularly, to a photobioreactor for a large-scale microalgal culture which uses a surface light source such as a light emitting diode (LED) element, an organic light emitting diode (OLED) element, or a flexible LED sheet, which has a thin thickness and high power consumption efficiency, in order to overcome a limitation to only a two-dimensional scaling-up due to a limitation of the transmittance of light in the event of a scaling-up.

2. Description of the Related Art

Various useful, high value-added substances derived from microalgae have been found throughout the world. A high concentration culture is indispensable for large-scale production of these substances. Culture systems currently being used are mainly large-scale cultures using outdoor culture facilities such as a large pond, which causes various problems. These problems include, for example, pollution, a low cell density which makes separation and purification difficult, irregular luminosity and climate conditions, and the requirements of a wide culture area, labor costs, a large amount of matrix (particularly, a nitrogen source), a high quality of water, and so on.

Particularly, in terms of the pollution problem, perdition caused by various protozoa, depletion of the matrix caused by photosynthetic microorganisms or other microorganisms, etc. are very serious, and require a separate expensive apparatus for concentrating products. Thus, culture technologies studied at each country in the world are focused primarily on producing products in excess of a throughput of the case of using a large pond in the quantitative aspect by using an indoor high-density culture photobioreactor having a relatively small size, and simultaneously producing a high quality of products in high concentration.

However, in the case of a routine microorganism culture apparatus, it is possible to quantitatively increase the throughput by means of three-dimensional scaling-up. In contrast, in the case of a high-density culture, since a limitation associated with the transmittance of light always exists, it is possible to quantitatively increase the throughput only by means of two-dimensional scaling-up. In detail, in the case of the photobioreactor, a ratio of the surface area of a light source to the volume of a reaction space must be kept high. Nevertheless, since the distance which light travels is limited, only the two-dimensional scaling-up in a direction parallel to a surface of the light source is possible.

Examples of this photobioreactor include an agitated reactor, a planar reactor, a tubular reactor, a columnar reactor, and so on. These reactors have difficulty in scaling-up, so that a new, large reactor is required for a large-scale culture.

In order to solve this problem, a “modular photobioreactor used for a large-scale microalgal culture” is disclosed in Korean Patent Publication No. 2003-0018197. In this document, as illustrated in FIGS. 1 and 2, the photobioreactor 100 includes a first reaction chamber 111 and a second reaction chamber 112, which are separated from each other such that parts, particularly lower portions, thereof communicate with each other. The first reaction chamber 111 has a medium injection port 113 which extends inwardly from the upper portion of a side wall thereof so as to supply nutrients to microalgae contained therein. The second reaction chamber 112 has a medium discharge port 114, which extends outwardly from the upper portion of a side wall thereof so as to discharge the medium injected through the medium injection port 113. Further, each of the first and second reaction chambers 111 and 112 has gas inflow ports 115, each of which extends downward from the bottom thereof so as to supply gas to the corresponding reaction chamber. In contrast, each of the first and second reaction chambers 111 and 112 has a gas outflow port 116, which extends upward from the top thereof so as to provide a path for exhausting the gas passing through the corresponding reaction chamber to the outside.

Further, for the purpose of a large-scale culture, a plurality of photobioreactors 100 can be set so as to be connected with other in parallel. At this time, the two neighboring photobioreactors 100 are connected with each other in such a manner that the medium discharge port 114 of one 100 of them is inserted into the medium injection port 113 of the other one 100.

As the photobioreactors 100 are assembled in this structure, the medium is injected through the medium injection port 113 of the first photobioreactor 100, nutrients are supplied to the microalgae contained in the first and second reaction chambers, and these are then discharged through the medium discharge port 114 of the first photobioreactor 100. Subsequently, the medium is injected into the second photobioreactor 100 through the medium injection port 113 of the second photobioreactor 100. The medium passing through the photobioreactor in this way is discharged to the outside through the medium discharge port 114 of the last photobioreactor 100.

As the gas injected through the gas inflow ports 115, air can be used. When it is necessary to supply carbon dioxide (CO₂), gas mixed with an excessive amount of carbon dioxide (between 5% and 20%) can be used. For example, this gas can use combustion exhaust gas. The gas passing through the photobioreactors is exhausted through the gas outflow port 116.

Light sources 117 such as fluorescent lamps are installed in a space between the first reaction chamber 111 and the second reaction chamber 112. The fluorescent lamps 117 are installed in such a manner that they are fitted with typical lamp shades or are merely stacked. The number of light sources is adjusted so as to control luminosity.

However, in the modular photobioreactor having the aforementioned structure, for the large-scale culture, the light receiving area is maintained, but the expansion of a volume is inevitable. In the case in which the fluorescent lamps are used as internal light sources, it is variously observed that the luminosity is reduced under the influence of a culture medium and a surrounding temperature (low temperature). Due to the volume of each fluorescent lamp itself, its application is also reduced.

Further, although the injected gas is mixed with the excessive amount of carbon dioxide (between 5% and 20%), a distance which the mixture gas passes, that is a flow distance between the gas inflow port 115 and the gas outflow port 116 of each reaction chamber, is limited, so that only an amount ranging from about 10% to about 20% of the carbon dioxide supplied for the reaction with the microalgae is used in each reaction chamber. Consequently, the modular photobioreactor shows low efficiency.

### SUMMARY OF THE INVENTION

Accordingly, the present invention has been made in an effort to solve the problems occurring in the related art, and embodiments of the present invention provide a photobioreactor for a large-scale microalgal culture, which overcomes a limitation of the transmittance of light to permit three-dimensional scaling-up, which applies a light source emitting a high intensity of light and reduces a burden on the volume so as to be suitable for scaling-up and increasing productivity per unit volume, and which allows supplied carbon dioxide to be used for reacting at a high ratio through increasing the flow distance of the supplied mixture gas so as to improve efficiency.

According to one aspect of the present invention, there is provided a photobioreactor for a large-scale microalgal culture, which comprises: a reaction tank containing microalgae to be cultured which has first and second walls disposed in parallel to each other in a symmetrical shape, a gas inflow port at a predetermined position of the first wall, and a gas outflow port at a predetermined position of the first or second wall spaced apart from the gas inflow port; and one or more surface-light-source assemblies installed so as to partition an interior space of the reaction tank in such a manner that the surface-light-source assemblies are disposed in the reaction tank between the gas inflow and outflow ports at predetermined intervals and are alternately in contact with the first and second walls, emitting light for culturing the microalgae in an internally illuminated fashion, and serving as a partition increasing a flow distance between the gas inflow and outflow ports. The photobioreactor permits three-dimensional scaling-up.

The reaction tank may have the shape of a cuboid such that the first and second walls serve as lower and upper walls respectively. Each surface-light-source assembly may have the shape of a flat plate.

Each surface-light-source assembly may be disposed so as to be inclined with respect to a vertical direction at a predetermined angle.

The reaction tank may have the shape of a cuboid such that the first and second walls serve as two lateral walls facing each other, respectively. Each surface-light-source assembly may have the shape of a flat plate.

Each surface-light-source assembly may be disposed so as to be inclined to a horizontal direction at a predetermined angle.

The reaction tank may have the shape of a cylinder including the first and second walls serving as circular upper and lower walls respectively and a cylindrical wall connecting the first and second walls with each other. The surface-light-source assemblies may have the shape of a cylinder and be disposed in the interior space of the reaction tank in a form concentric to the shape of the cylindrical wall.

The reaction tank may include at least one gas inflow port in an edge of the first wall, and a gas outflow port in a center of the first or second wall.

The interior space of the reaction tank which is partitioned by the surface-light-source assemblies may have intervals A gradually increasing outwards from the center to the edge of each wall.

Each surface-light-source assembly may include a flat or cylindrical board, a plurality of light emitting diode elements installed on one or opposite surfaces of the board under a rule of predetermined arrangement, and waterproof coating layers covering the opposite surfaces of the board on which the plurality of light emitting diode elements are installed.

Each surface-light-source assembly may include a flat or cylindrical board, a plurality of organic light emitting diode elements installed on one or opposite surfaces of the board under a rule of predetermined arrangement, and waterproof coating layers covering the opposite surfaces of the board on which the plurality of organic light emitting diode elements are installed.

Each surface-light-source assembly may include a flat or cylindrical board, a plurality of flexible light emitting diode sheets installed on one or opposite surfaces of the board under a rule of predetermined arrangement, and waterproof coating layers covering the opposite surfaces of the board on which the plurality of flexible light emitting diode sheets are installed.

According to embodiments of the present invention, the photobioreactor for a large-scale microalgal culture permits three-dimensional scaling-up, applies each surface-light-source assembly using a light emitting diode (LED) element, an organic light emitting diode (OLED) element, or a flexible LED sheet, which emits a high intensity of light and remarkably reduces a volume thereof, as a light source, so that it can facilitate scaling-up and increase productivity per unit volume. Further, the photobioreactor allows supplied carbon dioxide to be used for reacting at a high ratio by increasing the distance the supplied mixture gas flows, so that it can improve efficiency. Furthermore, the photobioreactor can reduce power consumption and operation expenses. Thus, the photobioreactor makes possible a remarkable easing of the spatial limitation, improvements in efficiency, and reduction in operation expenses, so that it can be very suitable for the large-scale culture of microalgae.

### BRIEF DESCRIPTION OF THE DRAWINGS

FIG. 1 is a perspective view illustrating a conventional modular photobioreactor having fluorescent lamps;

FIG. 2 is a side cross-sectional view illustrating reaction equipment in which the numerous modular photobioreactors of FIG. 1 are connected to each other;

FIG. 3 is a schematic perspective view illustrating a photobioreactor for a large-scale microalgal culture according to a first embodiment of the present invention;

FIG. 4 is a vertical cross-sectional view of the photobioreactor of FIG. 3;

FIG. 5 is a horizontal cross-sectional view of the photobioreactor of FIG. 3;

FIGS. 6 through 10 are cross-sectional views illustrating the cross-sectional structure of surface-light-source assemblies constituting a photobioreactor for a large-scale microalgal culture according to the present invention;

FIG. 11 is a vertical cross-sectional view illustrating a photobioreactor for a large-scale microalgal culture according to a second embodiment of the present invention;

FIG. 12 is a vertical cross-sectional view illustrating a photobioreactor for a large-scale microalgal culture according to a third embodiment of the present invention; and

FIG. 13 is a horizontal cross-sectional view of the photobioreactor of FIG. 12.

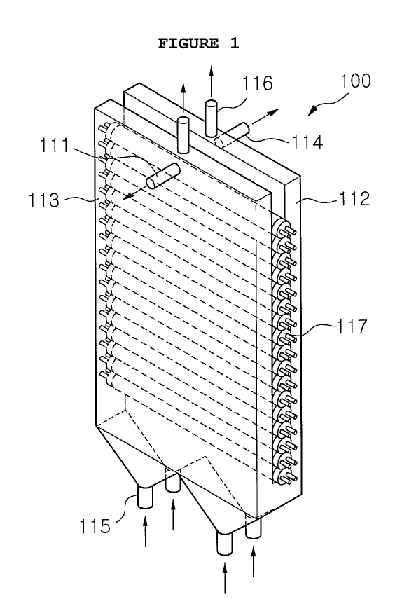

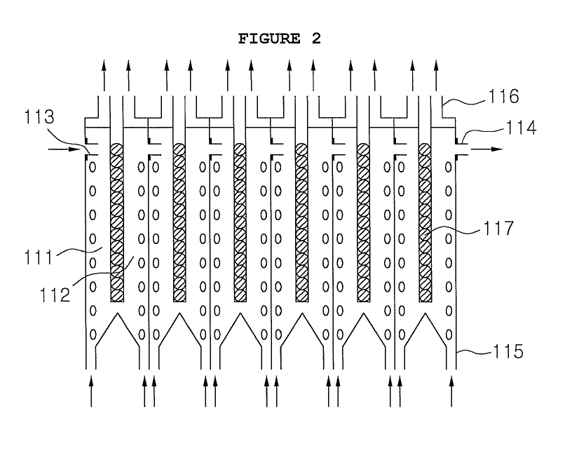

### DESCRIPTION OF THE PREFERRED EMBODIMENTS

Reference will now be made in greater detail to exemplary embodiments of the invention with reference to the accompanying drawings.

A photobioreactor for a large-scale microalgal culture according to a first embodiment of the present invention includes a reaction tank 10 and a plurality of surface-light-source assemblies 20.

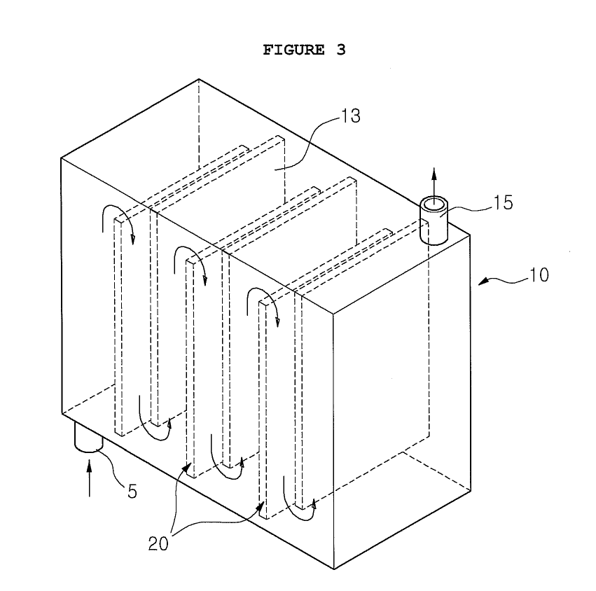

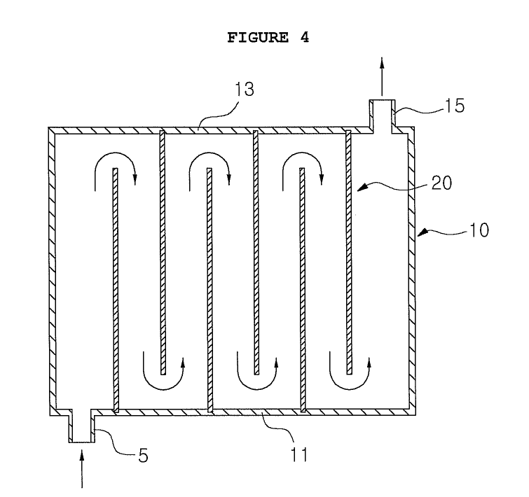

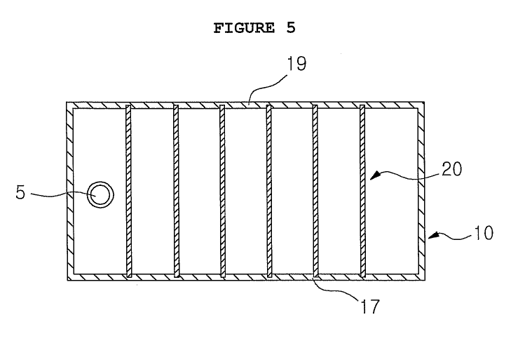

As illustrated in FIGS. 3 through 5, the reaction tank 10 is for containing microalgae to be cultured, and has the shape of a cuboid. Further, the reaction tank 10 includes a gas inflow port 5 in the left-hand corner of a lower wall 11 thereof, and a gas outflow port 15 in the right-hand corner of an upper wall 13 thereof spaced apart from the gas inflow port 5.

The surface-light-source assemblies 20, each of which has the shape of a flat plate, are installed in the reaction tank 10. More specifically, the surface-light-source assemblies 20 are installed so as to partition an interior space of the reaction tank 10 in such a manner that they are disposed between the gas inflow and outflow ports 5 and 15 of the reaction tank 10 at regular intervals, and are alternately in contact with the upper and lower walls 13 and 11 of the reaction tank 10, and emit light for culturing the microalgae in an internally illuminated fashion. In detail, the surface-light-source assemblies 20 having the shape of a flat plate are in contact with a total of three walls, these including front and rear walls 17 and 19 of the reaction tank 10 (which are contacted in common) and the upper or lower wall 13 or 11 of the reaction tank 10 (which is alternately contacted). These surface-light-source assemblies 20 are installed in the reaction tank 10 in a vertical direction. If necessary, the surface-light-source assemblies 20 may be installed so as to be inclined in the vertical direction at a predetermined angle.

As the surface-light-source assemblies 20 are installed in this way, they serve as partitions that remarkably increase a flow distance between the gas inflow port 5 and the gas outflow port 15. Further, the photobioreactor makes possible a two-dimensional scaling-up in a direction parallel to the surfaces of each surface-light-source assembly 20 as well as a three-dimensional scaling-up in a direction perpendicular to the surfaces of each surface-light-source assembly 20 in addition to the direction parallel to the surfaces of each surface-light-source assembly 20, thereby increasing dimensions of the reaction tank, and thus installing more surface-light-source assemblies 20. In this manner, the photobioreactor is very suitable for the scaling-up.

Although not separately illustrated, in the case in which the last surface-light-source assembly is installed so as to be in contact with the lower wall 11 of the reaction tank 10 depending on a variation in the number of surface-light-source assemblies 20, the gas outflow port 15 may be formed in the front right-hand corner of the lower wall 11 of the reaction tank 10.

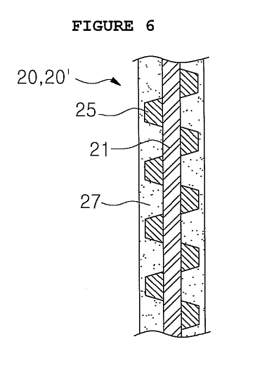

Each surface-light-source assembly 20 functions to emit light for culturing the microalgae. As illustrated in FIGS. 6 through 10, each surface-light-source assembly 20 uses, as a light source, a light emitting diode (LED) element 25, an organic light emitting diode (OLED) element 35, or a flexible LED sheet 45, which emits a strong intensity of light with a small volume, so that it can on the whole remarkably reduce a burden on the volume.

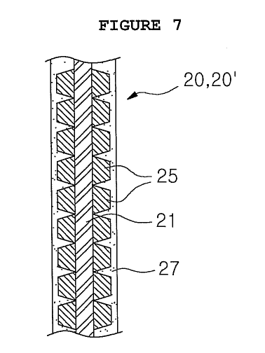

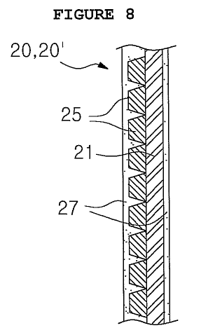

In detail, as illustrated in FIGS. 6 through 8, each surface-light-source assembly 20 can be composed of a flat board 21, a plurality of LED elements 25 installed on one or opposite surfaces of the board 21 under the rule of predetermined arrangement, and waterproof coating layers 27 covering the opposite surfaces of the board 21 on which the plurality of LED elements 25 are installed. As illustrated in FIGS. 6 and 7, each surface-light-source assembly 20 can be configured so that the LED elements 25 are installed on all of the opposite surfaces of the board 21. As illustrated in FIG. 8, each surface-light-source assembly 20 can be configured so that the LED elements 25 are installed on one of the opposite surfaces of the board 21. Further, as illustrated in FIG. 6, each surface-light-source assembly 20 can be configured so that the LED elements 25 are installed on the board 21 spaced apart from each other at regular intervals under a predetermined rule. As illustrated in FIG. 7, each surface-light-source assembly 20 can be configured so that the LED elements 25 are installed on the board 21 so as to be dense enough to cover the entire board 21.

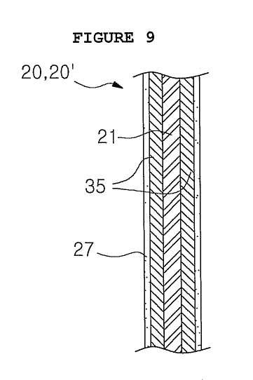

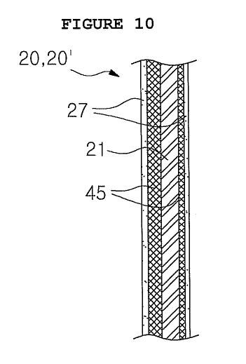

Meanwhile, as illustrated in FIG. 9, each surface-light-source assembly 20 can use the OLED elements 35 instead of the LED elements 25. As illustrated in FIG. 10, each surface-light-source assembly 20 can use the flexible LED sheets 45 instead of the LED elements 25.

The photobioreactor for a large-scale microalgal culture according to a second embodiment of the present invention includes a reaction tank 10 and a plurality of surface-light-source assemblies 20, as in the first embodiment.

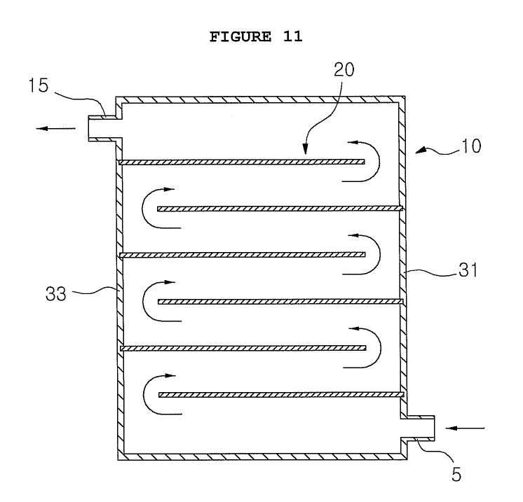

As illustrated in FIG. 11, the reaction tank 10 contains microalgae to be cultured, and has the shape of a cuboid. However, unlike in the first embodiment, the reaction tank 10 includes a gas inflow port 5 in the lower corner of a right-hand wall 31 thereof, and a gas outflow port 15 in the upper corner of a left-hand wall 33 thereof spaced apart from the gas inflow port 5.

The surface-light-source assemblies 20, each of which has the shape of a flat plate, are installed in the reaction tank 10. In detail, the surface-light-source assemblies 20 are installed so as to partition an interior space of the reaction tank 10 in such a manner that they are disposed between the gas inflow and outflow ports 5 and 15 of the reaction tank 10 at regular intervals, and are alternately in contact with the right-hand and left-hand walls 31 and 33 of the reaction tank 10, and emit light for culturing the microalgae in an internally illuminated fashion. In detail, the surface-light-source assemblies 20 having the shape of a flat plate are in contact with the total of three walls, these walls including front and rear walls of the reaction tank 10 which are contacted in common, and the right-hand or left-hand wall 31 or 33 of the reaction tank 10 with which alternating contact is made. These surface-light-source assemblies 20 are installed in the reaction tank 10 in a horizontal direction. If necessary, the surface-light-source assemblies 20 may be installed so as to be inclined with respect to the horizontal direction at a predetermined angle.

As the surface-light-source assemblies 20 are installed in this way, they serve as partitions that remarkably increase a flow distance between the gas inflow port 5 and the gas outflow port 15. Further, the photobioreactor makes possible a two-dimensional scaling-up in a direction parallel to the surfaces of each surface-light-source assembly 20 as well as a three-dimensional scaling-up in a direction perpendicular to the surfaces of each surface-light-source assembly 20 in addition to the direction parallel to the surfaces of each surface-light-source assembly 20, thereby increasing dimensions of the reaction tank, and thus installing more surface-light-source assemblies 20. In this manner, the photobioreactor is very suitable for scaling-up.

Although not separately illustrated, in the case in which the last surface-light-source assembly is installed so as to be in contact with the right-hand wall 31 of the reaction tank 10 depending on a variation in the number of surface-light-source assemblies 20, the gas outflow port 15 may be formed in the upper corner of the right-hand wall 31 of the reaction tank 10.

In this manner, the photobioreactor for a large-scale microalgal culture according to the second embodiment will be easily understood when it is regarded as a structure in which the transversely disposed photobioreactor for a large-scale microalgal culture according to the first embodiment is disposed in a longitudinal direction.

The photobioreactor for a large-scale microalgal culture according to a third embodiment of the present invention includes a reaction tank 10′ and a plurality of surface-light-source assemblies 20′ as in the first and second embodiments.

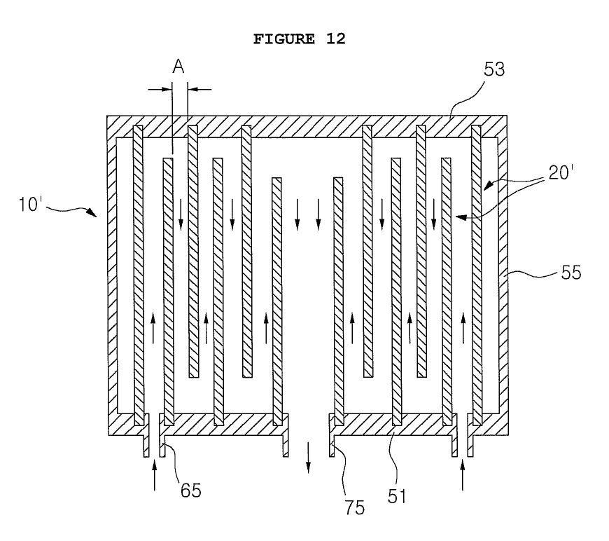

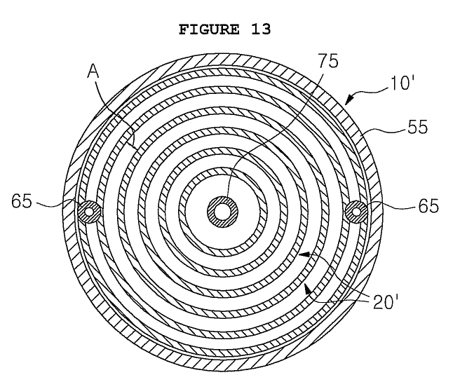

However, as illustrated in FIGS. 12 and 13, the reaction tank 10′ has the shape of a cylinder. In detail, the reaction tank 10′ includes a circular lower wall 51, a circular upper wall 53, and a cylindrical wall 55 connecting the upper and lower walls 51 and 53 with each other. Further, the reaction tank 10′ includes two gas inflow ports 65 on diagonal opposite edges of the lower wall 51 thereof, and a gas outflow port 75 in the center of the lower wall 51 spaced apart from the gas inflow ports 65.

The surface-light-source assemblies 20′ are installed in the reaction tank 10′ in a cylindrical shape. In other words, the surface-light-source assemblies 20′ are disposed in the reaction tank 10′ in the form concentric to the shape of the cylindrical wall 55. More specifically, the surface-light-source assemblies 20′ having different diameters are installed so as to partition an interior space of the reaction tank 10′ in such a manner that they have the center in common and are disposed from the edge where each gas inflow port 65 is located to the center where the gas outflow port 75 is located at regular intervals, and that they are alternately in contact with the lower and upper walls 51 and 53 of the reaction tank 10′, and emit light for culturing the microalgae in an internally illuminated fashion. Consequently, the surface-light-source assemblies 20′ having the shape of a cylinder are alternately in contact with the lower and upper walls 51 and 53 of the reaction tank 10′. At this time, preferably, each interval A of the interior space of the reaction tank partitioned by the surface-light-source assemblies 20′, i.e. each interval A between the neighboring surface-light-source assemblies 20′ is gradually increasing outwards from the center to the edge of the reaction tank in consideration of a cross section of the flow.

As the surface-light-source assemblies 20′ are installed in this way, they serve as partitions that remarkably increase a flow distance between each gas inflow port 65 and the gas outflow port 75. Further, the photobioreactor makes possible a two-dimensional scaling-up in a direction parallel to the surfaces of each surface-light-source assembly 20′ as well as a three-dimensional scaling-up in a direction perpendicular to the surfaces of each surface-light-source assembly 20 in addition to the direction parallel to the surfaces of each surface-light-source assembly 20, thereby increasing the diameter of the reaction tank, and thus installing more surface-light-source assemblies 20. In this manner, the photobioreactor is very suitable for scaling-up.

Although not separately illustrated, in the case in which the surface-light-source assembly 20′ having the smallest diameter is installed so as to be in contact with the upper wall 53 of the reaction tank 10′ depending on a variation in the number of surface-light-source assemblies 20′, the gas outflow port 75 may be formed in the center of the upper wall 53 of the reaction tank 10′.

In comparison with the first and second embodiments, the third embodiment has the same configuration except that a detailed cross-sectional structure of each surface-light-source assembly 20′ is a cylindrical structure for the board 21 rather than a flat plate structure, and so an additional detailed description will be omitted.

Although not separately illustrated, each photobioreactor for a large-scale microalgal culture as described above is basically equipped with medium injection and discharge ports for supplying nutrients to the microalgae at predetermined positions of the reaction tank, in addition to the gas inflow port 5 or 65 and the gas outflow port 15 or 75.

According to the embodiments of the present invention, the surface-light-source assemblies 20 or 20′ of the photobioreactor for a large-scale microalgal culture are not limited to the specific shapes as illustrated in FIGS. 6 through 10, but they can be variously modified. For example, different types of OLED elements 35 may be mounted on the opposite surfaces of the board 21 so as to emit light having different luminosities or reduce a difference between wavelengths. This modification makes it possible to create various culture environments. Further, the surface-light-source assemblies 20 or 20′ can variously adjust the luminosity, if various on-off controls are made by adding a control circuit (not shown) to a power supply means used to supply electric power. Thus, a larger quantity of microalgae can be cultured within a preset scale. Although the density of the microalgae increases during the culturing period, the intensity of the light is adjusted, and thus a quantity of light can be appropriately maintained. Since the light can be adjusted to various intensities, various culture requirements can be met.

According to embodiments of the present invention, the photobioreactor for a large-scale microalgal culture makes possible a two-dimensional scaling-up in a direction parallel to the surfaces of each surface-light-source assembly as well as a three-dimensional scaling-up in a direction perpendicular to the surfaces of each surface-light-source assembly in addition to the direction parallel to the surfaces of each surface-light-source assembly, so that it is very suitable for scaling-up.

Further, the photobioreactor uses a surface light source such as an LED element, an OLED element, or a flexible LED element having a small volume and high power-consumption efficiency, as compared to a conventional photobioreactor using a light source such as a fluorescent lamp having a large volume and low power-consumption efficiency, so that it can configure each flat or cylindrical surface-light-source assembly 20 at a very thin thickness of about several millimeters. As a result, the photobioreactor can not only be advantageous for the scaling-up due to easing of spatial limitations, but also can remarkably improve productivity per unit volume.

In addition, as described above, the photobioreactor allows carbon dioxide contained in mixture gas to be used for reacting at a high ratio by increasing the distance of the mixture gas flows in the interior space of the reaction tank, so that it can remarkably improve efficiency.

In sum, the photobioreactor for a large-scale microalgal culture makes possible a remarkable easing of the spatial limitations, improvements of efficiency associated with the supply of gas, and a reduction in operation expenses related to power consumption, and can meet various culture requirements to expand an applicable range, so that it can be very suitable for the large-scale culture of microalgae.

1. A photobioreactor for a large-scale microalgal culture, comprising:
a reaction tank containing microalgae to be cultured and having first and second walls disposed in parallel to each other in a symmetrical shape, a gas inflow port at a predetermined position of the first wall, and a gas outflow port at a predetermined position of the first or second wall spaced apart from the gas inflow port; and
one or more surface-light-source assemblies installed so as to partition an interior space of the reaction tank in such a manner that the surface-light-source assemblies are disposed in the reaction tank between the gas inflow and outflow ports at predetermined intervals and are alternately in contact with the first and second walls, emitting light for culturing the microalgae in an internally illuminated fashion, and serving as a partition increasing a flow distance between the gas inflow and outflow ports,
wherein the photobioreactor permits three-dimensional scaling-up.

2. The photobioreactor as set forth in claim 1, wherein:
the reaction tank has a shape of a cuboid such that the first and second walls serve as lower and upper walls respectively; and
each surface-light-source assembly has a shape of a flat plate.

3. The photobioreactor as set forth in claim 2, wherein each surface-light-source assembly is disposed so as to be inclined with respect to a vertical direction at a predetermined angle.

4. The photobioreactor as set forth in claim 1, wherein:
the reaction tank has a shape of a cuboid such that the first and second walls serve as two lateral walls facing each other respectively; and
each surface-light-source assembly has a shape of a flat plate.

5. The photobioreactor as set forth in claim 4, wherein each surface-light-source assembly is disposed so as to be inclined with respect to a horizontal direction at a predetermined angle.

6. The photobioreactor as set forth in claim 1, wherein:
the reaction tank has a shape of a cylinder comprising first and second walls serving as circular upper and lower walls respectively and a cylindrical wall connecting the first and second walls with each other; and
the surface-light-source assemblies have a shape of a cylinder and are disposed in the interior space of the reaction tank in a shape concentric to that of the cylindrical wall.

7. The photobioreactor as set forth in claim 6, wherein the reaction tank includes at least one gas inflow port in an edge of the first wall, and the gas outflow port in a center of the first or second wall.

8. The photobioreactor as set forth in claim 7, wherein the interior space of the reaction tank which is partitioned by the surface-light-source assemblies has intervals (A) gradually increasing outwards from the center to the edge of each wall.

9. The photobioreactor as set forth in claim 1, wherein each surface-light-source assembly comprises a flat or cylindrical board, a plurality of light emitting diode elements installed on one or opposite surfaces of the board under a rule of predetermined arrangement, and waterproof coating layers covering the opposite surfaces of the board on which the plurality of light emitting diode elements are installed.

10. The photobioreactor as set forth in claim 1, wherein each surface-light-source assembly comprises a flat or cylindrical board, a plurality of organic light emitting diode elements installed on one or opposite surfaces of the board under a rule of predetermined arrangement, and waterproof coating layers covering the opposite surfaces of the board on which the plurality of organic light emitting diode elements are installed.

11. The photobioreactor as set forth in claim 1, wherein each surface-light-source assembly comprises a flat or cylindrical board, a plurality of flexible light emitting diode sheets installed on one or opposite surfaces of the board under a rule of predetermined arrangement, and waterproof coating layers covering the opposite surfaces of the board on which the plurality of flexible light emitting diode sheets are installed.
